SEQUENCE LISTING (8 sequences)

SEQ ID NO: 1 - DNA, 33 bp, human
cggtagaattcaatatttgcatgtcgctatgtg

SEQ ID NO: 2 - DNA, 46 bp, human
gaagagcggatccgctcttctttttaagagtggtctcatacagaac

SEQ ID NO: 3 - DNA, 72 bp, human
tatgcttaccgtaacttgaaagtatttcgatttcttggctttatatatcttgtggaaaaaacgagaagagcg

SEQ ID NO: 4 - DNA, 74 bp, human
gatccgctcttctcgttttttccacaagatatataaagccaagaaatcgaaatactttcaagttacggtaagca

SEQ ID NO: 5 - DNA, 22 bp, Photinus
acgtgcgttgctagtaccaact

SEQ ID NO: 6 - DNA, 22 bp, Photinus
aaaagttggtactagcaacgca

SEQ ID NO: 7 - DNA, 373 bp, human
gggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaaaacgagaagagcggatccgctcttctttttaagagtggtctcatacagaacttataagattcccaaatccaaagacatttcacgtttatggtgatttcccagaacacatagcgacatgcaaatatt

SEQ ID NO: 8 - DNA, 48 bp, Bacteriophage T7
ctagcataaccccttggggcctctaaacgggtcttgaggggttttttg

(19) United States
(12) Patent Application Publication
(10) Pub. No.: US 2005/0060771 A1
(43) Pub. Date: Mar. 17, 2005

(54) siRNA encoding constructs and methods for using the same

(76) Inventor: Andrew Alan Farmer, Mountain View, CA (US)

Correspondence Address: BOZICEVIC, FIELD & FRANCIS (BD BIOSCIENCES), 1900 UNIVERSITY AVENUE, SUITE 200, EAST PALO ALTO, CA, 94303, US

(21) Appl. No.: 10/927,962

(22) Filed: Aug. 27, 2004

Related U.S. Application Data
(60) Provisional application No. 60/502,403, filed on Sep. 11, 2003.

(51) Int. Cl. A01H 1/00; C12N 15/82

(52) U.S. Cl. 800/286; 435/468

(57) ABSTRACT

siRNA encoding constructs and methods for using the same are provided. The subject constructs are characterized by including a siRNA coding domain flanked by opposing promoters. In using the subject constructs, sense and antisense strands of the desired siRNA product encoded by the coding domain are transcribed under the direction of the two opposing promoters flanking the coding domain. The transcribed sense and antisense strands are then annealed to each other to produce the desired siRNA double-stranded molecule. The subject constructs and methods find use in a variety of applications, including applications where the specific reduction or silencing of a gene is desired. Also provided are systems and kits for use in practicing the subject invention.

### CROSS-REFERENCE To RELATED APPLICATIONS

Pursuant to 35 U.S.C. § 119 (e), this application claims priority to the filing date of U.S. Provisional Patent Application Ser. No. 60/502,403 filed Sep. 11, 2003; the disclosure of which is herein incorporated by reference.

### INTRODUCTION

### BACKGROUND OF THE INVENTION

RNAi has become the method of choice for loss-of-function investigations in numerous systems including, C. elegans, Drosophila, fungi, plants, and even mammalian cell lines. To specifically silence a gene in most mammalian cell lines, small interfering RNAs (siRNA) are used because large dsRNAs (>30 bp) trigger the interferon response and cause nonspecific gene silencing. Currently, siRNAs are produced by chemical synthesis, by in vitro transcription from a short DNA template, or by transfection of DNA plasmids that give rise to hairpin RNAs in vivo.

In many applications to date, a vector is employed to deliver into the target environment, e.g., cell, an expression cassette that encodes hairpin RNAs. The vector may be a DNA plasmid or other suitable vector, e.g., viral vector. However, in these approaches, long oligos are required for construction of hairpin sequences, where these long oligos can be expensive and difficult to make. In addition, the requisite long oligos are harder to clone and are not so readily usable for library construction.

As such, there is a continued need for the development of new protocols for use in delivering an RNAi agent to a target environment. The present invention satisfies this need.

### RELEVANT LITERATURE

U.S. Pat. Nos. 6,547,099; 6,506,559; and 4,766,072. Published U.S. Application No. 20020006664; 20030153519; 20030139363. Published PCT applications of interest include WO 01/68836 and WO 03/010180. See also: Bernstein, et al., Nature (2001) 409:363-366; Brummelkamp, et al., Science (2002) 296:550-553; Elbashir, et al., Genes Dev. (2001)15(2):188-200; Fire et al., Nature (1998) 391:806-811; Hammond et al., Nature Rev. Gen. (2001) 2:110-119; Harborth et al., J. Cell Science (2001)114:4557-4565; Hutvagner & Zamore, Curr. Opin. Genetics & Development (2002) 12:225-232; Kennerdell & Carthew, Cell (1998) 95:1017-1026; Lee et al., Nature Biotechnol. (2002) 20:500-505; Nykanen et al., Cell (2001)107:309-321; Paddison et al, Genes & Dev. (2002)16:948-958; Paul et al., Nature Biotechnol. (2002) 20:505-508; Scherr et al., Blood (2002) 101(4):1566-1569; Sharp, P. A. Genes Dev. (2001) 15:485-490; Sui et al., Proc. Natl. Acad. Sci. USA (2002) 99(8):5515-5520; Wang et al., J. Biol. Chem. (2000) 275:40174-40179; Yu et al. Proc. Natl. Acad. Sci. USA (2002) 99(9):6047-6052.

### SUMMARY OF THE INVENTION

siRNA encoding constructs and methods for using the same are provided. The subject constructs are characterized by including a siRNA coding domain flanked by opposing promoters. In using the subject constructs, sense and antisense strands of the desired siRNA product encoded by the coding domain are transcribed under the direction of the two opposing promoters flanking the coding domain. The transcribed sense and antisense strands are then annealed to each other to produce the desired siRNA double-stranded product molecule. The subject constructs and methods find use in a variety of applications, including applications where the specific reduction or silencing of a gene is desired. Also provided are systems and kits for use in practicing the subject invention.

In certain embodiments, the invention provides a construct that has two opposing promoters flanking a siRNA coding domain, wherein each of the promoters includes a transcription terminator, e.g., that is present in a non-transcribed region of each of said promoters. In certain embodiments, the transcription terminator is adjacent to the siRNA coding domain, e.g., wherein the distance between the last nucleotide of the coding domain and the first nucleotide of the terminator is less than about 20 nt, such as less than about 10 nt, e.g, less than about 5 nt, where the distance between the last nucleotide of the coding domain and the first nucleotide of the terminator may range from about 0 to about 5 nt. In certain embodiments, the siRNA coding domain is made up of deoxyribonucleotides. In certain embodiments, the coding domain encodes a siRNA double stranded molecule that is between about 20 and about 30 bp in length. In certain embodiments, at least one of the two opposing promoters is an inducible promoter. In certain embodiments, the construct is present on a vector, e.g., a plasmid or a viral vector.

Also provided are methods of producing a siRNA double-stranded molecule. Such methods include transcribing sense and anstisense RNA strands from a construct as described above. In certain embodiments, the methods are in vitro methods, while in other embodiments the methods are in vivo methods. In certain embodiments, the methods further include a step of making the employed constructs, e.g., vectors, e.g., by first providing a provector that includes two opposing promoters flanking at least one cloning or insertion site, and then introducing the coding domain into said cloning site. The cloning site may be a multiple cloning site and/or may include a sequence cleaved by a type IIS restriction endonuclease.

Also provided are methods of making a construct that encodes a siRNA double-stranded molecule, e.g., by introducing a coding sequence for the siRNA double-stranded molecule into a cloning site of a proconstruct, wherein the cloning site is flanked by two opposing promoters, wherein each of said promoters includes a transcription terminator. In certain embodiments, the transcription terminator is present in a non-transcribed region of each of said promoters, e.g., adjacent to the siRNA coding domain. In certain embodiments, the distance between the last nucleotide of the coding domain and the first nucleotide of the terminator is less than about 20 nt, e.g., less than about 10 nt, including less than about 5 nt. In certain embodiments, the distance between the last nucleotide of the coding domain and the first nucleotide of the terminator ranges from about 0 to about 5 nt. In certain embodiments, the cloning site is a multiple cloning site and/or includes at least one sequence cleaved by a type IIS restriction endonuclease. In certain embodiments, at least one of the two opposing promoters is an inducible promoter.

Also provided are methods of at least reducing the expression of a gene in a target cell. Such methods include introducing into the cell an effective amount of construct having two opposing promoters flanking a siRNA coding domain, wherein each of the promoters includes a transcription terminator, e.g., as described above. In certain embodiments, the methods are in vivo methods, e.g., and may be methods of silencing expression of a gene, such as in a loss of function assay.

Also provided are proconstructs as described above. Also provided are kits, where the kits may include a proconstruct of the invention, and instructions for inserting a siRNA coding domain into the insertion site of the proconstruct. In certain embodiments, the kits further include a restriction endonuclease for the insertion site. Also provided are systems for use in preparing RNAi double-stranded molecules, where the systems include a proconstruct and a restriction endonuclease for the cloning site thereof, as described above.

### BRIEF DESCRIPTION OF THE FIGURES

FIG. 1 provides a schematic of a structure of an RNAi coding construct according to an embodiment of the subject invention.

FIG. 2 provides a map of a representative embodiment of a vector construct according to the present invention, e.g., the dual U6-H1 promoter construct.

FIG. 3 provides the sequence of above vector shown in FIG. 2.

### DEFINITIONS

For convenience, certain terms employed in the specification, examples, and appended claims are collected here.

As used herein, the term “vector” refers to a nucleic acid molecule capable of transporting another nucleic acid to which it has been linked. One type of vector is a genomic integrated vector, or “integrated vector”, which can become integrated into the chromosomal DNA of the host cell. Another type of vector is an episomal vector, i.e., a nucleic acid capable of extra-chromosomal replication in an appropriate host, e.g., a eukaryotic or prokaryotic host cell. Vectors capable of directing the expression of genes to which they are operatively linked are referred to herein as “expression vectors”.

As used herein, the term “nucleic acid” refers to polynucleotides such as deoxyribonucleic acid (DNA), and, where appropriate, ribonucleic acid (RNA). The term should also be understood to include, as applicable to the embodiment being described, single-stranded (such as sense or antisense) and double-stranded polynucleotides.

As used herein, the term “gene” or “recombinant gene” refers to a nucleic acid comprising an open reading frame encoding a polypeptide of the present invention, including both exon and (optionally) intron sequences. A “recombinant gene” refers to nucleic acid encoding such regulatory polypeptides, that may optionally include intron sequences that are derived from chromosomal DNA. The term “intron” refers to a DNA sequence present in a given gene that is not translated into protein and is generally found between exons. As used herein, the term “transfection” means the introduction of a nucleic acid, e.g., an expression vector, into a recipient cell by nucleic acid-mediated gene transfer.

A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from procaryotic or eukaryotic mRNA, genomic DNA sequences from procaryotic or eukaryotic DNA, and even synthetic DNA sequences. A transcription termination sequence will usually be located 3′ to the coding sequence.

Likewise, “encodes”, unless evident from its context, will be meant to include DNA sequences that encode a polypeptide, as the term is typically used, as well as DNA sequences that are transcribed into inhibitory antisense and RNAi molecules.

By “reducing expression” is meant that the level of expression of a target gene or coding sequence is reduced or inhibited by at least about 2-fold, usually by at least about 5-fold, e.g., 10-fold, 15-fold, 20-fold, 50-fold, 100-fold or more, as compared to a control. By modulating expression of a target gene is meant altering, e.g., reducing, transcription/translation of a coding sequence, e.g., genomic DNA, mRNA etc., into a polypeptide, e.g., protein, product.

The term “expression” with respect to a gene sequence refers to transcription of the gene and, as appropriate, translation of the resulting mRNA transcript to a protein. Thus, as will be clear from the context, expression of a protein coding sequence results from transcription and translation of the coding sequence.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, the term “transfection” is art recognized and means the introduction of a nucleic acid, e.g., an expression vector, into a recipient cell by nucleic acid-mediated gene transfer. “Transduction”, as used herein, refers to a process in which a cell's genotype is changed as a result of the cellular uptake of exogenous DNA or RNA, and, for example, the transformed cell expresses a dsRNA construct. “Transient transfection” refers to cases where exogenous DNA does not integrate into the genome of a transfected cell, e.g., where episomal DNA is transcribed into mRNA and translated into protein.

A cell has been “stably transfected” with a nucleic acid construct when the nucleic acid construct is capable of being inherited by daughter cells.

As used herein, a “reporter gene construct” is a nucleic acid that includes a “reporter gene” operatively linked to at least one transcriptional regulatory sequence. Transcription of the reporter gene is controlled by these sequences to which they are linked. The activity of at least one or more of these control sequences can be directly or indirectly regulated by the target receptor protein. Exemplary transcriptional control sequences are promoter sequences. A reporter gene is meant to include a promoter-reporter gene construct that is heterologously expressed in a cell.

“Inhibition of gene expression” refers to the absence (or observable decrease) in the level of protein and/or mRNA product from a target gene. “Specificity” refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

Depending on the assay, quantitation of the amount of gene expression allows one to determine a degree of inhibition which is greater than 10%, 33%, 50%, 90%, 95% or 99% as compared to a cell not treated according to the present invention. Lower doses of administered active agent and longer times after administration of active agent may result in inhibition in a smaller fraction of cells (e.g., at least 10%, 20%, 50%, 75%, 90%, or 95% of targeted cells). Quantitation of gene expression in a cell may show similar amounts of inhibition at the level of accumulation of target mRNA or translation of target protein. As an example, the efficiency of inhibition may be determined by assessing the amount of gene product in the cell: mRNA may be detected with a hybridization probe having a nucleotide sequence outside the region used for the inhibitory double-stranded RNA, or translated polypeptide may be detected with an antibody raised against the polypeptide sequence of that region.

### DESCRIPTION OF THE SPECIFIC EMBODIMENTS

siRNA encoding constructs and methods for using the same are provided. The subject constructs are characterized by including a siRNA coding domain flanked by, and operationally linked to, opposing promoters. In using the subject constructs, sense and antisense strands of the desired siRNA encoded by the coding domain are transcribed under the direction of the two opposing promoters flanking the coding domain. The transcribed sense and antisense strands are then annealed to each other to produce the desired siRNA double-stranded product molecule. The subject constructs and methods find use in a variety of applications, including applications where the specific reduction or silencing of a gene is desired. Also provided are systems and kits for use in practicing the subject invention.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Although any methods, devices and materials similar or equivalent to those described herein can be used in the practice or testing of the invention, representative methods, devices and materials are now described.

All publications mentioned herein are incorporated herein by reference for the purpose of describing and disclosing the components that are described in the publications which might be used in connection with the presently described invention.

In further describing the subject invention, the subject constructs and vectors containing the same are described first in greater detail, followed by a review of methods of using the subject constructs/vectors as well as representative applications therefore, followed by a review of representative systems and kits according to the present invention.

Constructs and Vectors

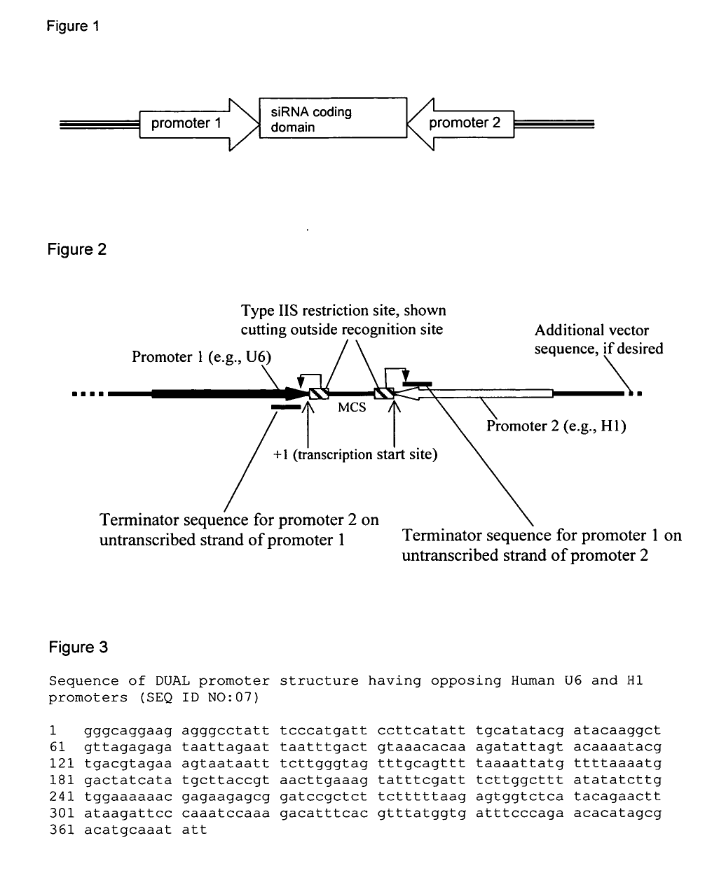

As summarized above, the subject invention provides constructs and vectors that can be used, as described in more detail below, to produce double stranded siRNA molecules. The subject constructs are characterized, in the broadest sense, by including a double stranded deoxyribonucleic acid domain that includes a siRNA coding domain flanked by, and operationally linked to, opposing promoters. In other words, the constructs include a double-stranded DNA region that has the structure depicted in FIG. 1. As such, in the subject constructs, the siRNA coding domain is bounded on either side by a promoter, where the promoters have directions or polarity facing each other such that when the promoters are activated to cause transcription of the adjacent operationally linked coding domain, they each transcribe one of the strands of the siRNA coding domain, i.e., either the sense or antisense strand.

The siRNA coding domain of the present invention is one that encodes a siRNA product, where the product is a double-stranded ribonucleic acid molecule that is made up of two annealed RNA strands, e.g., sense and antisense strands, that are not covalently bound to each other. The siRNA product molecules in many embodiments are made up of annealed RNA strands that range in length from about 10 to about 30-35 residues, e.g., from about 15 to about 25 residues, including from about 20 to 23 residues, where molecules of 12, 15, 18, 20, 21, 22, 25 and 29 residues in length are of particular interest in certain embodiments. As such, the length of the siRNA coding domain typically ranges from about 10 to about 30-35 bp, such as from about 15 to about 25 bp, including from about 20 to about 23 bp.

Flanking either side of the above siRNA coding domain is a promoter. The distance between the promoter and the coding domain may vary, so long as the distance is not so great such that the transcribed sense and antisense strands do not fail to anneal to produce the desired siRNA, or that the product does not perform its intended function, as described in greater detail below. In certain embodiments, the promoter is immediately adjacent to the coding domain, such that there are no intervening nucleotides. In yet other embodiments, the promoter is separated from the coding domain by a short intervening sequence, e.g., from about 1 to about 30 bp, such as from about 1 to about 20 bp, including from about 1 to about 10 bp, where in certain embodiments, the distance does not exceed about 8, 6, 4 or 2 bp.

In general, any convenient promoter may be employed as the first and second promoters, so long as the promoters can be activated in the desired environment to transcribe their operatively linked strand of the siRNA coding domain and produce the desired sense or antisense strand. The promoters may be the same or different, and in certain embodiments are different. The promoters may be constitutive or inducible, as desired. Exemplary promoters for use in the present invention are selected such that they are functional in the cell type (and/or animal or plant) into which they are being introduced. Specific promoters of interest include promoters that have defined transcriptional starts sites and relatively short terminators. Representative specific promoters of interest include, but are not limited to: pol III promoters (such as mammalian (e.g., mouse or human) U6 and H1 promoters, VA1 promoters, tRNA promoters, etc.); pol II promoters; inducible promoters, e.g., TET inducible promoters; bacteriophage RNA polymerase promoters, e.g., T7, T3 and Sp6, and the like. Other promoters known in the art may also be employed, where the particular promoters chosen will depend, at least in part, on the environment in which expression is desired. Specific promoter pairings of interest include, but are not limited to any paired combination of the above specifically listed promoters (i.e., U6, H1, VA1, tRNA, TET, T7, T3, Sp6).

The first and second flanking promoters are also modified so that they have transcription terminators located in their non-transcribed strands (regions) relative to the opposing promoter (see FIG. 2), so as to allow termination of the transcription being driven by the opposing promoter immediately after the coding domain without also including the termination sequence in the expressed strand. As such, the promoters are characterized by including a transcription terminator, i.e., a termination sequence, at their terminus adjacent to the coding domain, where the transcription terminator is in frame and operatively linked to the sequence transcribed by the opposing promoter, such that it serves to terminate transcription of the coding domain under control of the opposing promoter. The distance between the last nucleotide of the coding domain and the first nucleotide of the terminator is, in certain embodiments, less than about 20 nt, such as less than about 10 nt, including less than about 5 nt, ranging from about 0 to about 5 nt, such as from about 0 to about 3 nt, including from about 0 to about 2 nt. Transcription terminator sequences are well known in the art, e.g., about 4-5 T adjacent T residues, CTAGCATAACCCCTTGGGGCCTCTAAACGGGTCTTGAGGGGTTTTTTG (T7 terminator) (SEQ ID NO:08), etc.

The above constructs made up of a siRNA coding domain flanked by two opposing promoters are, in many embodiments, present on a vector. The constructs may be present on any convenient type of vector, where representative vectors of interest include, but are not limited to: plasmid vectors, viral vectors, and the like.

Representative eukaryotic plasmid vectors of interest include, for example: pCMVneo, pShuttle, pDNR and Ad-X (Clontech Laboratories, Inc.); as well as BPV, EBV, vaccinia, SV40, 2-micron circle, pcDNA3.1, pcDNA3.1/GS, pYES2/GS, pMT, p IND, pIND(SpI), pVgRXR, and the like, or their derivatives. Such plasmids are well known in the art (Botstein et al., Miami Wntr. SyTnp. 19:265-274, 1982; Broach, In: “The Molecular Biology of the Yeast Saccharomyces: Life Cycle and Inheritance”, Cold Spring Harbor Laboratory, Cold Spring Harbor, N.Y., p. 445-470, 1981; Broach, Cell 28:203-204, 1982; Dilon et at., J. Clin. Hematol. Oncol. 10:39-48, 1980; Maniatis, In: Cell Biology: A Comprehensive Treatise, Vol. 3, Gene Sequence Expression, Academic Press, NY, pp. 563-608,1980.

A variety of viral vector delivery vehicles are known to those of skill in the art and include, but are not limited to: adenovirus, herpesvirus, lentivirus, vaccinia virus and adeno-associated virus (AAV).

The above described constructs and vectors may be produced using any convenient protocol. In many embodiments, a proconstruct is employed to produce the above-described vectors and constructs. The proconstruct is characterized by having two opposing promoters flanking an insertion site, where the insertion site includes one or more sequences recognized and cleaved by a restriction endonuclease. In certain embodiments, the insertion site includes a single recognized sequence, while in other embodiments, the insertion site includes multiple different restriction endonuclease recognized sequences, e.g., it is a multiple cloning site. While any convenient restriction endonuclease recognized sequence may be used for the insertion site, (and a multitude of such sequences are known in the art), of interest in many embodiments are sites recognized or cleaved by Type IIS restriction endonucleases. Representative known Type IIS restriction endonuncleases are SapI, bbsI, BsaI.

When using such proconstructs, the insertion site is first cleaved with the appropriate endonuclease, e.g., the appropriate Type IIS endonuclease. The desired siRNA coding sequence is then cloned into the site, e.g., using standard protocols.

In certain embodiments, the proconstruct may include a selectable marker located in the cloning site. In these embodiments, if a coding sequence is succesfully introduced into the cloning site, the selectable marker is ablated and absence of signal confirms successful integration of the coding sequence. Any convenient marker or reporter sequence may be employed. For example, the activity of the reporter genes like lacZ or GFP may be monitored by measuring a detectable signal (e.g., fluorescent or chromogenic) that results from the activation of these reporter genes. For example, lacZ translation can be monitored by incubation in the presence of a chromogenic substrate, such as X-gal (5-bromo-4-chloro-3-indolyl-β-D-galactoside), for its encoded enzyme, β-galactosidase.

The above-mentioned procedures, e.g., of cleavage, plasmid construction, etc., are well known to one skilled in the art and the enzymes required for restriction and ligation are available commercially. (See, for example, R. Wu, Ed., Methods in Enzymology, Vol. 68, Academic Press, N.Y. (1979); T. Maniatis, E. F. Fritsch and J. Sambrook, Molecular Cloning: A Laboratory Manual, Cold Spring Harbor Laboratory Press, Cold Spring Harbor, N.Y. (1982); Catalog 1982-83, New England Biolabs, Inc.; Catalog 1982-83, Bethesda Research Laboratories, Inc.

Methods

Also provided are methods of producing double-stranded RNAi molecules using the above-described constructs and vectors. In general, the subject methods include transcribing both strands of the siRNA coding domain to produce two complementary RNA strands, which strands are then allowed to anneal to each other to produce the desired double stranded siRNA product molecule. As such, in practicing the subject methods, the above constructs are maintained in an environment in which the promoters direct transcription of their respective operatively linked strands of the coding domain. Where the promoters are constitutive, transcription may occur at the same time from both promoters. In other embodiments, e.g., where one of the promoters is inducible, transcription will proceed upon induction of the promoter(s) and may or may not occur at the same time in both directions.

Production of the siRNA product molecules according to the present methods may occur in a cell free environment or inside of a cell. Where production of the RNAi product molecules is desired to occur inside of a cell, any convenient method of delivering the construct to the target cell may be employed. Where it is desired to produce the siRNA molecules inside of a cell, the above construct is introduced into the target cell. Any convenient protocol may be employed, where the protocol may provide for in vitro or in vivo introduction of the construct into the target cell, depending on the location of the target cell.

For example, where the target cell is an isolated cell, the construct may be introduced directly into the cell under cell culture conditions permissive of viability of the target cell, e.g., by using standard transformation techniques. Such techniques include, but are not necessarily limited to: viral infection, transformation, conjugation, protoplast fusion, electroporation, particle gun technology, calcium phosphate precipitation, direct microinjection, viral vector delivery, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

Alternatively, where the target cell or cells are part of a multicellular organism, the construct may be administered to the organism or host in a manner such that the construct is able to enter the target cell(s), e.g., via an in vivo or ex vivo protocol. By “in vivo,” it is meant in the target construct is administered to a living body of an animal. By “ex vivo” it is meant that cells or organs are modified outside of the body. Such cells or organs are typically returned to a living body. Methods for the administration of nucleic acid constructs are well known in the art. Nucleic acid constructs can be delivered with cationic lipids (Goddard, et al, Gene Therapy, 4:1231-1236,1997; Gorman, et al, Gene Therapy 4:983-992,1997; Chadwick, et al, Gene Therapy 4:937-942, 1997; Gokhale, et al, Gene Therapy 4:1289-1299, 1997; Gao, and Huang, Gene Therapy 2:710-722, 1995), using viral vectors (Monahan, et al, Gene Therapy 4:40-49, 1997; Onodera, et al, Blood 91:30-36, 1998), by uptake of “naked DNA”, and the like. Techniques well known in the art for the transformation of cells (see discussion above) can be used for the ex vivo administration of nucleic acid constructs. The exact formulation, route of administration and dosage can be chosen empirically. (See e.g. Fingl et al., 1975, in “The Pharmacological Basis of Therapeutics”, Ch. 1 p1).

As such, in certain embodiments the construct, including constructs present on vectors (e.g., plasmids, viral vectors, etc) are administered to a multicellular organism that includes the target cell. By multicellular organism is meant an organism that is not a single celled organism. Multicellular organisms of interest include animals, where animals of interest include vertebrates, where the vertebrate is a mammal in many embodiments. Mammals of interest include; rodents, e.g. mice, rats; livestock, e.g. pigs, horses, cows, etc., pets, e.g. dogs, cats; and primates, e.g. humans.

The selected route of administration of the construct to the multicellular organism depends on several parameters, including: the nature of the vectors that carry the construct, the nature of the delivery vehicle, the nature of the multicellular organism, and the like. In certain embodiments, linear or circularized DNA, e.g. a plasmid, is employed as the vector for delivery of the construct to the target cell. In such embodiments, the plasmid may be administered in an aqueous delivery vehicle, e.g. a saline solution. Alternatively, an agent that modulates the distribution of the vector in the multicellular organism may be employed. For example, where the vectors comprising the subject system components are plasmid vectors, lipid based, e.g. liposome, vehicles may be employed, where the lipid based vehicle may be targeted to a specific cell type for cell or tissue specific delivery of the vector. Patents disclosing such methods include: U.S. Pat. Nos. 5,877,302; 5,840,710; 5,830,430; and 5,827,703, the disclosures of which are herein incorporated by reference. Alternatively, polylysine based peptides may be employed as carriers, which may or may not be modified with targeting moieties, and the like. (Brooks, A. I., et al. 1998, J. Neurosci. Methods V. 80 p: 137-47; Muramatsu, T., Nakamura, A., and H. M. Park 1998, Int. J. Mol. Med. V. 1 p: 55-62). In yet other embodiments, the construct may be incorporated onto viral vectors, such as adenovirus derived vectors, sindbis virus derived vectors, retroviral derived vectors, etc. hybrid vectors, and the like, as described above. The above vectors and delivery vehicles are merely representative. Any vector/delivery vehicle combination may be employed, so long as it provides for the desired introduction of the construct in into the target cell.

As such, in vivo and in vitro gene therapy delivery of the constructs according to the present invention is also encompassed by the present invention. In vivo gene therapy may be accomplished by introducing the construct into cells via local injection of a polynucleotide molecule or other appropriate delivery vectors. (Hefti, J. Neurobiology, 25:1418-1435, 1994). For example, a polynucleotide molecule including the construct may be contained in an adeno-associated virus vector for delivery to the targeted cells (See for e.g., International Publication No. WO 95/34670; International Application No. PCT/US95/07178). The recombinant adeno-associated virus (AAV) genome typically contains AAV inverted terminal repeats flanking a DNA sequence that includes the construct.

Alternative viral vectors include, but are not limited to, retrovirus, adenovirus, herpes simplex virus and papilloma virus vectors. U.S. Pat. No. 5,672,344 (issued Sep. 30, 1997, Kelley et al., University of Michigan) describes an in vivo viral-mediated gene transfer system involving a recombinant neurotrophic HSV-1 vector. U.S. Pat. No. 5,399,346 (issued Mar. 21, 1995, Anderson et al., Department of Health and human Services) provides examples of a process for providing a patient with a therapeutic protein by the delivery of human cells which have been treated in vitro to insert a DNA segment encoding a therapeutic protein. Additional methods and materials for the practice of gene therapy techniques are described in U.S. Pat. No. 5,631,236 (issued May 20, 1997, Woo et al., Baylor College of Medicine) involving adenoviral vectors; U.S. Pat. No. 5,672,510 (issued Sep. 30, 1997, Eglitis et al., Genetic Therapy, Inc.) involving retroviral vectors; and U.S. Pat. No. 5,635,399 (issued Jun. 3, 1997, Kriegler et al., Chiron Corporation) involving retroviral vectors expressing cytokines.

Nonviral delivery methods include liposome-mediated transfer, naked DNA delivery (direct injection), receptor-mediated transfer (ligand-DNA complex), electroporation, calcium phosphate precipitation and microparticle bombardment (e.g., gene gun). Gene therapy materials and methods may also include inducible promoters, tissue-specific enhancer-promoters, DNA sequences designed for site-specific integration, DNA sequences capable of providing a selective advantage over the parent cell, labels to identify transformed cells, negative selection systems and expression control systems (safety measures), cell-specific binding agents (for cell targeting), cell-specific internalization factors, transcription factors to enhance expression by a vector as well as methods of vector manufacture. Such additional methods and materials for the practice of gene therapy techniques are described in U.S. Pat. No. 4,970,154 (issued Nov. 13, 1990, D. C. Chang, Baylor College of Medicine) electroporation techniques; International Application No. WO 9640958 (published 961219, Smith et al., Baylor College of Medicine) nuclear ligands; U.S. Pat. No. 5,679,559 (issued Oct. 21, 1997, Kim et al., University of Utah Research Foundation) concerning a lipoprotein-containing system for gene delivery; U.S. Pat. No. 676,954 (issued Oct. 14, 1997, K. L. Brigham, Vanderbilt University involving liposome carriers; U.S. Pat. No. 5,593,875 (issued Jan. 14, 1997, Wurm et al., Genentech, Inc.) concerning methods for calcium phosphate transfection; and U.S. Pat. No. 4,945,050 (issued Jul. 31, 1990, Sanford et al., Cornell Research Foundation) wherein biologically active particles are propelled at cells at a speed whereby the particles penetrate the surface of the cells and become incorporated into the interior of the cells. Expression control techniques include chemical induced regulation (e.g., International Application Nos. WO 9641865 and WO 9731899), the use of a progesterone antagonist in a modified steroid hormone receptor system (e.g., U.S. Pat. No. 5,364,791), ecdysone control systems (e.g., International Application No. WO 9637609), and positive tetracycline-controllable transactivators (e.g., U.S. Pat. Nos. 5,589,362; 5,650,298; and 5,654,168).

Because of the multitude of different types of vectors and delivery vehicles that may be employed, administration may be by a number of different routes, where representative routes of administration include: oral, topical, intraarterial, intravenous, intraperitoneal, intramuscular, etc. The particular mode of administration depends, at least in part, on the nature of the delivery vehicle employed for the vectors which harbor the construct. In certain embodiments, the vector or vectors harboring the construct are administered intravascularly, e.g. intraarterially or intravenously, employing an aqueous based delivery vehicle, e.g. a saline solution.

Utility

The above-described constructs and ds RNAi producs produced therefrom find use in a variety of different applications. Representative applications include, but are not limited to: drug screening/target validation, large scale functional library screening, silencing single genes, silencing families of genes, e.g., ser/thr kinases, phosphatases, membrane receptors, etc., and the like. The subject constructs and products thereof also find use in therapeutic applications, as described in greater detail separately below.

One representative utility of the present invention is as a method of identifying gene function in an organism, especially higher eukaryotes using the product siRNA to inhibit the activity of a target gene of previously unknown function. Instead of the time consuming and laborious isolation of mutants by traditional genetic screening, functional genomics using the subject product siRNA determines the function of uncharacterized genes by employing the siRNA to reduce the amount and/or alter the timing of target gene activity. The product siRNA can be used in determining potential targets for pharmaceutics, understanding normal and pathological events associated with development, determining signaling pathways responsible for postnatal development/aging, and the like. The increasing speed of acquiring nucleotide sequence information from genomic and expressed gene sources, including total sequences for mammalian genomes, can be coupled with use of the product siRNA to determine gene function in a cell or in a whole organism. The preference of different organisms to use particular codons, searching sequence databases for related gene products, correlating the linkage map of genetic traits with the physical map from which the nucleotide sequences are derived, and artificial intelligence methods may be used to define putative open reading frames from the nucleotide sequences acquired in such sequencing projects.

A simple representative assay inhibits gene expression according to the partial sequence available from an expressed sequence tag (EST). Functional alterations in growth, development, metabolism, disease resistance, or other biological processes would be indicative of the normal role of the ESTs gene product

The ease with which the construct encoding the siRNA product can be introduced into an intact cell/organism containing the target gene allows the present invention to be used in high throughput screening (HTS). For example, individual clones from the library can be replicated and then isolated in separate reactions, or the library is maintained in individual reaction vessels (e.g., a 96 well microtiter plate) to minimize the number of steps required to practice the invention and to allow automation of the process. Solutions containing the constructs or product siRNAs thereof that are capable of inhibiting the different expressed genes can be placed into individual wells positioned on a microtiter plate as an ordered array, and intact cells/organisms in each well can be assayed for any changes or modifications in behavior or development due to inhibition of target gene activity.

The constructs or siRNA products thereof can be fed directly to, injected into, the cell/organism containing the target gene. The constructs or siRNA products may be directly introduced into the cell (i.e., intracellularly); or introduced extracellularly into a cavity, interstitial space, into the circulation of an organism, introduced orally, or may be introduced by bathing an organism in a solution containing the constructs or siRNA products. Methods for oral introduction include direct mixing of nucleic acids with food of the organism. Physical methods of introducing nucleic, acids include injection directly into the cell or extracellular injection into the organism of a nucleic acid solution. The constructs or siRNA products thereof may be introduced in an amount which allows delivery of at least one copy per cell. Higher doses (e.g., at least 5, 10, 100, 500 or 1000 copies per cell) of constructs or products thereof may yield more effective inhibition; lower doses may also be useful for specific applications. Inhibition is sequence-specific in that nucleotide sequences corresponding to the duplex region of the RNA are targeted for genetic inhibition.

The function of the target gene can be assayed from the effects it has on the cell/organism when gene activity is inhibited. This screening could be amenable to small subjects that can be processed in large number, for example, tissue culture cells derived from invertebrates or invertebrates, mammals, especially primates, and most preferably humans.

If a characteristic of an organism is determined to be genetically linked to a polymorphism through RFLP or QTL analysis, the present invention can be used to gain insight regarding whether that genetic polymorphism might be directly responsible for the characteristic. For example, a fragment defining the genetic polymorphism or sequences in the vicinity of such a genetic polymorphism can be screened for its impact, e.g., by producing a siRNA molecule corresponding to the fragment in the organism or cell, and evaluating whether an alteration in the characteristic is correlated with inhibition.

The present invention is useful in allowing the inhibition of essential genes. Such genes may be required for cell or organism viability at only particular stages of development or cellular compartments. The functional equivalent of conditional mutations may be produced by inhibiting activity of the target gene when or where it is not required for viability. The invention allows addition of siRNA at specific times of development and locations in the organism without introducing permanent mutations into the target genome.

In situations where alternative splicing produces a family of transcripts that are distinguished by usage of characteristic exons, the present invention can target inhibition through the appropriate exons to specifically inhibit or to distinguish among the functions of family members. For example, a hormone that contained an alternatively spliced transmembrane domain may be expressed in both membrane bound and secreted forms. Instead of isolating a nonsense mutation that terminates translation before the transmembrane domain, the functional consequences of having only secreted hormone can be determined according to the invention by targeting the exon containing the transmembrane domain and thereby inhibiting expression of membrane-bound hormone.

Therapeutic Applications

The subject constructs and siRNA products thereof also find use in a variety of therapeutic applications in which it is desired to selectively modulate, e.g., one or more target genes in a host, e.g., whole mammal, or portion thereof, e.g., tissue, organ, etc, as well as in cells present therein. In such methods, an effective amount of the subject constructs or products thereof is administered to the host or target portion thereof. By effective amount is meant a dosage sufficient to selectively modulate expression of the target gene(s), as desired. As indicated above, in many embodiments of this type of application, the subject methods are employed to reduce/inhibit expression of one or more target genes in the host or portion thereof in order to achieve a desired therapeutic outcome.

Depending on the nature of the condition being treated, the target gene may be a gene derived from the cell, an endogenous gene, a pathologically mutated gene, e.g. a cancer causing gene, one or more genes whose expression causes or is related to heart disease, lung disease, alzheimer's disease, parkinson's disease, diabetes, arthritis, etc.; a transgene, or a gene of a pathogen which is present in the cell after infection thereof, e.g., a viral (e.g., HIV-Human Immunodeficiency Virus; HBV-Hepatitis B virus; HCV-Hepatitis C virus; Herpes-simplex 1 and 2; Varicella Zoster (Chicken pox and Shingles); Rhinovirus (common cold and flu); any other viral form) or bacterial pathogen. Depending on the particular target gene and the dose of construct or siRNA product delivered, the procedure may provide partial or complete loss of function for the target gene. Lower doses of injected material and longer times after administration of siRNA may result in inhibition in a smaller fraction of cells.

The subject methods find use in the treatment of a variety of different conditions in which the modulation of target gene expression in a mammalian host is desired. By treatment is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

The present invention is not limited to modulation of expression of any specific type of target gene or nucleotide sequence. Representative classes of target genes of interest include but are not limited to: developmental genes (e.g., adhesion molecules, cyclin kinase inhibitors, cytokines/lymphokines and their receptors, growth/differentiation factors and their receptors, neurotransmitters and their receptors); oncogenes (e.g., ABL1, BCL1, BCL2, BCL6, CBFA2, CBL, CSFIR, ERBA, ERBB, EBRB2, ETS1, ETS1, ETV6, FOR, FOS, FYN, HCR, HRAS, JUN, KRAS, LCK, LYN, MDM2, MLL, MYB, MYC, MYCL1, MYCN, NRAS, PIM 1, PML, RET, SRC, TAL1, TCL3, and YES); tumor suppressor genes (e.g., APC, BRCA 1, BRCA2, MADH4, MCC, NF 1, NF2, RB 1, TP53, and WT1); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, Upases, lipoxygenases, lyso/ymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases); chemokines (e.g. CXCR4, CCR5), the RNA component of telomerase, vascular endothelial growth factor (VEGF), VEGF receptor, tumor necrosis factors nuclear factor kappa B, transcription factors, cell adhesion molecules, Insulin-like growth factor, transforming growth factor beta family members, cell surface receptors, RNA binding proteins (e.g. small nucleolar RNAs, RNA transport factors), translation factors, telomerase reverse transcriptase); etc.

As indicated above, the constructs or siRNA products thereof can be introduced into the target cell(s) using any convenient protocol, where the protocol will vary depending on whether the target cells are in vitro or in vivo.

Where the target cells are in vivo, the constructs or siRNA products thereof can be administered to the host comprising the cells using any convenient protocol, where the protocol employed is typically a nucleic acid administration protocol, where a number of different such protocols are known in the art. The following discussion provides a review of representative nucleic acid administration protocols that may be employed. The nucleic acids may be introduced into tissues or host cells by any number of routes, including microinjection, or fusion of vesicles. Jet injection may also be used for intra-muscular administration, as described by Furth et al. (1992), Anal Biochem 205:365-368. The nucleic acids may be coated onto gold microparticles, and delivered intradermally by a particle bombardment device, or “gene gun” as described in the literature (see, for example, Tang et al. (1992), Nature 356:152-154), where gold microprojectiles are coated with the DNA, then bombarded into skin cells.

For example, the constructs or siRNA products thereof can be fed directly to, injected into, the host organism containing the target gene. The agent may be directly introduced into the cell (i.e., intracellularly); or introduced extracellularly into a cavity, interstitial space, into the circulation of an organism, introduced orally, etc. Methods for oral introduction include direct mixing of RNA with food of the organism. Physical methods of introducing nucleic acids include injection directly into the cell or extracellular injection into the organism of an RNA solution.

In certain embodiments, a hydrodynamic nucleic acid administration protocol is employed. Where the agent is a ribonucleic acid, the hydrodynamic ribonucleic acid administration protocol described in detail below is of particular interest. Where the agent is a deoxyribonucleic acid, the hydrodynamic deoxyribonucleic acid administration protocols described in Chang et al., J. Virol. (2001) 75:3469-3473; Liu et al., Gene Ther. (1999) 6:1258-1266; Wolff et al., Science (1990) 247: 1465-1468; Zhang et al., Hum. Gene Ther. (1999) 10:1735-1737: and Zhang et al., Gene Ther. (1999) 7:1344-1349; are of interest.

Additional nucleic acid delivery protocols of interest include, but are not limited to: those described in U.S. Patents of interest include U.S. Pat. Nos. 5,985,847 and 5,922,687 (the disclosures of which are herein incorporated by reference); WO/11092; Acsadi et al., New Biol. (1991) 3:71-81; Hickman et al., Hum. Gen. Ther. (1994) 5:1477-1483; and Wolff et al., Science (1990) 247: 1465-1468; etc. See e.g., the viral and non-viral mediated delivery protocols described above.

Depending on the nature of the constructs or siRNA products thereof, the active agent(s) may be administered to the host using any convenient means capable of resulting in the desired modulation of target gene expression. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the agents can be achieved in various ways, including oral, buccal, rectal, parenteral, intraperitoneal, intradermal, transdermal, intracheal, etc., administration.

In pharmaceutical dosage forms, the agents may be administered alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill in the art will readily appreciate that dose levels can vary as a function of the specific compound, the nature of the delivery vehicle, and the like. Preferred dosages for a given compound are readily determinable by those of skill in the art by a variety of means.

Systems

Also provided are systems for practicing one or more of the above-described methods. In certain embodiments, the systems are systems for producing constructs that can be used to produce the siRNA products described above. Such systems typically include a proconstruct, a corresponding restriction endonuclease, and a siRNA coding domain, as described above. In yet other embodiments, the proconstruct may be precut, such that the corresponding enzyme is not included. In certain embodiments, the systems are systems for producing a siRNA double stranded molecule, as described above. In such embodiments, the systems include a construct, e.g., present on a vector, as described above, and any other reagents desirable for transcribing the the sense and antisense strands from the vector to produce the desired siRNA product, where representative reagents include host cells, factors, etc.

Kits

Also provided are reagents and kits thereof for practicing one or more of the above-described methods. The subject reagents and kits thereof may vary greatly. In certain embodiments, the kits include at least a proconstruct, e.g., provector. In certain embodiments, the kits may further include a restriction endonuclease corresponding to the insertion site of the proconstruct (or a means for producing the same (such as an encoding nucleic acid)), a host cell, buffers, factors, etc. In yet other embodiments, the proconstruct may be precut or linearized, such that the restriction endonuclease is not included. In certain embodiments, the kits at least include the subject constructs, and any other reagents desirable for transcribing the the sense and antisense strands from the vector to produce the desired siRNA product, where representative reagents include host cells, factors, etc.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### EXPERIMENTAL

I. Construction and use of pSIREN_Retro-Q-Dual
A. Construction of pSIREN_Retro-Q-Dual
   The pSIREN_Retro-Q-Dual vector is built as follows:

The PSIREN-DNR (BD Bioscience Clontech part # S3424) is taken and digested with EcoRI and BamHI, according to standard practice. The Human H1 promoter is then amplified from a genomic DNA library (e.g., human Caucasian placental library: HL1067j; BD Biosciences Clontech) using the following (or similar) primers, based on primer design principals known in the art:

(SEQ ID NO:01)

Fwd primer for H1:
5′ CGGTAGAATTCAATATTTGCATGTCGCTATGTG

(SEQ ID NO:02)

Rev primer for H1:
5′ GAAGAGCGGATCCGCTCTTCTTTTTAAGAGTGGTCTCATACAGAAC

This action generates a PCR product containing the H1 promoter, flanked by a BamHI site at the 3′ end and an EcoRI site at the 5′ end. In addition, the H1 promoter is modified, such that a Pol III terminator sequence (TTTTT) is encoded starting at the transcription start site and continuing within the promoter's untranscribed region (i.e., 5′ of the transcription start site). In addition, a SapI site ((Type IIS restriction site) is added into the MC between the BamHI site and the Terminator. This SapI site is so positioned such that it will cut the DNA inside the promoter/terminator sequence—allowing the seemless cloning of gene-specific oligos for RNAi into the vector between the 2 promoters.

The PCR product is digested with BamHI and EcoRI, according to standard practice and is cloned into the previously digested pSIREN-DNR. This will generate the vector: pSIREN-DNR-Q-U6-H1.

The U6 promoter within the vector is then also modified to include a terminator upstream of the transcription start site as follows. pSIREN-DNR-U6-H1 is digested with BamHI and NdeI. The following oligos are then annealed, according to standard practice, and ligated into the digested pSIREN-DNR-U6-H1.

(SEQ ID NO:03)

U6 Modifying primer 1
5′ tatgcttaccgtaacttgaaagtatttcgatttcttggctttatata
tcttgtggaaaaaacgagaagagcg

(SEQ ID NO:04)

U6 modifying primer 2
5′ gatccgctcttctcgttttttccacaagatatataaagccaagaaat
cgaaatactttcaagttacggtaagca

This introduces the terminator sequence into the U6 promoter and additionally adds a second SapI site (Type IIS restriction site) in the MCS. Thus the vector now has in the following order:
U6 promoter—Terminator, SapI site, BamHI site, SapI site, Terminator, H1 promoter

Where the H1 promoter is oriented opposite to the U6 and where the Terminator in the H1 promoter is on the transcribed strand of the U6 promoter and the terminator in the U6 promoter is on the transcribed strand of the H1 promoter.

This Dual promoter cassette is then transferred to the pSIREN-RetroQ backbone (BD Bioscience Clontech cat #631526) as follows:

The pSIREN-RetroQ vector is Digested with BglII and EcoRI and the large fragment containing the vector backbone is retained. Similarly, pSIREN-DNR-U6-H1 is digested with BglII and EcoRI and the fragment carrying the U6 and H1 promoters is retained. These two fragments are then ligated together to generate PSIREN-RetroQ-U6-H1.

Finally, an undesired Sap I site in the backbone of this vector is removed by standard mutagenesis using the kit from stratagene (Quick Change mutagenesis kit: cat # 200518). This generates the final desired vector: pSIREN_Retro-Dual.

B. Use of Vector

To clone a siRNA expression oligo into this vector the following is done (example for luciferase)

An oligo pair encoding the Sense and antisense strands of the siRNA sequence are made that include overhangs matching those generated by Sap I digestion of pSIREN-Retro-Dual. These oligos are annealed and ligated into Sap I digested pSIREN-Retro-Dual to give a vector containing the sense strand of the siRNA under control of the U6 promoter and the antisense strand under control of the H1 promoter.

| | |
|---|---|
| Sense strand oligo: | |
| 5′ ACGTGCGTTGCTAGTACCAACT | (SEQ ID NO:05) |
| Antisense strand oligo: | |
| 5′ AAAAGTTGGTACTAGCAACGCA | (SEQ ID NO:06) |

To cause RNAi, this vector is then transfected using standard methods into cells expressing the gene to which the siRNA is designed.

It is evident from the above results and discussion that the subject invention provides improved methods of producing siRNAs, as well as improved methods of using the produced siRNAs in various applications, including high throughput loss of function applications. As such, the subject invention makes the rapid determination of gene function possible. Accordingly, the present invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A construct comprising two opposing promoters flanking a siRNA coding domain, wherein each of said promoters comprises a transcription terminator.

2. The construct according to claim 2, wherein said transcription terminator is present in a non-transcribed region of each of said promoters.

3. The construct according to claim 2, wherein in each of said promoters, said transcription terminator is adjacent to said siRNA coding domain.

4. The construct according to claim 3, wherein the distance between the last nucleotide of said coding domain and the first nucleotide of said terminator is less than about 20 nt.

5. The construct according to claim 4, wherein the distance between the last nucleotide of said coding domain and the first nucleotide of said terminator is less than about 10 nt.

6. The construct according to claim 5, wherein the distance between the last nucleotide of said coding domain and the first nucleotide of said terminator is less than about 5 nt.

7. The construct according to claim 6, wherein the distance between the last nucleotide of said coding domain and the first nucleotide of said terminator ranges from about 0 to about 5 nt.

8. The construct according to claim 1, wherein said siRNA coding domain comprises deoxyribonucleotides.

9. The construct according to claim 1, wherein said coding domain encodes a siRNA double stranded molecule that is between about 20 and about 30 bp in length.

10. The construct according to claim 1, wherein at least one of said two opposing promoters is an inducible promoter.

11. The construct according to claim 1, wherein said construct is present on a vector.

12. The construct according to claim 11, wherein said vector is a plasmid.

13. The construct according to claim 11, wherein said vector is a viral vector.

14. A method of producing a siRNA double-stranded molecule, said method comprising:
transcribing sense and anstisense RNA strands from a siRNA coding domain flanked by two opposing promoters so that said sense and antisense strands anneal to each other to produce said siRNA double-stranded molecule, wherein each of said promoters comprises a transcription terminator.

15. The method according to claim 14, wherein said transcription terminator is present in a non-transcribed region of each of said promoters.

16. The method according to claim 15, wherein in each of said promoters, said transcription terminator is adjacent to said siRNA coding domain.

17. The method according to claim 16, wherein the distance between the last nucleotide of said coding domain and the first nucleotide of said terminator is less than about 20 nt.

18. The method according to claim 17, wherein the distance between the last nucleotide of said coding domain and the first nucleotide of said terminator is less than about 10 nt.

19. The method according to claim 18, wherein the distance between the last nucleotide of said coding domain and the first nucleotide of said terminator is less than about 5 nt.

20. The method according to claim 19, wherein the distance between the last nucleotide of said coding domain and the first nucleotide of said terminator ranges from about 0 to about 5 nt.

21. The method according to claim 14, wherein said two opposing promoters flanking said coding sequence are present on a vector.

22. The method according to claim 21, wherein said vector is a plasmid.

23. The method according to claim 21, wherein said vector is a viral vector.

24. The method according to claim 14, wherein said method is an in vitro method.

25. The method according to claim 14, wherein said method is an in vivo method.

26. The method according to claim 14, further comprising making said vector by:
(a) providing a provector comprising said two opposing promoters flanking at least one cloning site, and
(b) introducing said coding domain into said cloning site.

27. The method according to claim 26, wherein said cloning site is a multiple cloning site.

28. The method according to claim 26, wherein said cloning site comprises a sequence cleaved by a type IIS restriction endonuclease.

29. The method according to claim 14, wherein at least one of said two opposing promoters is an inducible promoter.

30. A method of making a construct that encodes a siRNA double-stranded molecule, said method comprising:
introducing a coding sequence for said siRNA double-stranded molecule into a cloning site of a proconstruct, wherein said cloning site is flanked by two opposing promoters, wherein each of said promoters comprises a transcription terminator.

31. The method according to claim 30, wherein said transcription terminator is present in a non-transcribed region of each of said promoters.

32. The method according to claim 31, wherein in each of said promoters, said transcription terminator is adjacent to said siRNA coding domain.

33. The method according to claim 32, wherein the distance between the last nucleotide of said coding domain and the first nucleotide of said terminator is less than about 20 nt.

34. The method according to claim 33, wherein the distance between the last nucleotide of said coding domain and the first nucleotide of said terminator is less than about 10 nt.

35. The method according to claim 34, wherein the distance between the last nucleotide of said coding domain and the first nucleotide of said terminator is less than about 5 nt.

36. The method according to claim 35, wherein the distance between the last nucleotide of said coding domain and the first nucleotide of said terminator ranges from about 0 to about 5 nt.

37. The method according to claim 30, wherein said cloning site is a multiple cloning site.

38. The method according to claim 30, wherein said cloning site comprises at least one sequence cleaved by a type IIS restriction endonuclease.

39. The method according to claim 30, wherein at least one of said two opposing promoters is an inducible promoter.

40. A method of at least reducing the expression of a gene in a target cell, said method comprising:
introducing into said cell an effective amount of construct comprising two opposing promoters flanking a siRNA coding domain, wherein each of said promoters comprises a transcription terminator, under conditions sufficient to transcribe sense and anstisense RNA strands from said siRNA coding domain that anneal to each other to produce a siRNA double-stranded molecule for said gene that at least reduces expression of said gene.

41. The method according to claim 40, wherein said transcription terminator is present in a non-transcribed region of each of said promoters.

42. The method according to claim 41, wherein in each of said promoters, said transcription terminator is adjacent to said siRNA coding domain.

43. The method according to claim 42, wherein the distance between the last nucleotide of said coding domain and the first nucleotide of said terminator is less than about 20 nt.

44. The method according to claim 43, wherein the distance between the last nucleotide of said coding domain and the first nucleotide of said terminator is less than about 10 nt.

45. The method according to claim 44, wherein the distance between the last nucleotide of said coding domain and the first nucleotide of said terminator is less than about 5 nt.

46. The method according to claim 45, wherein the distance between the last nucleotide of said coding domain and the first nucleotide of said terminator ranges from about 0 to about 5 nt.

47. The method according to claim 40, wherein said construct is present on a vector.

48. The method according to claim 47, wherein said vector is a plasmid.

49. The method according to claim 47, wherein said vector is a viral vector.

50. The method according to claim 40, wherein said method is an in vitro method.

51. The method according to claim 40, wherein said method is an in vivo method.

52. The method according to claim 40, wherein said method is a method of silencing expression of said gene.

53. The method according to claim 40, wherein said method is a loss of function assay.

54. The method according to claim 40, wherein at least one of said two opposing promoters is an inducible promoter.

55. A proconstruct comprising:
two opposing promoters flanking an insertion site, wherein each of said promoters comprises a transcription terminator.

56. The proconstruct according to claim 55, wherein said transcription terminator is present in a non-transcribed region of each of said promoters.

57. The proconstruct according to claim 56, wherein in each of said promoters, said transcription terminator is adjacent to said insertion site.

58. The proconstruct according to claim 56, wherein the distance between the last nucleotide of said insertion site and the first nucleotide of said terminator is less than about 20 nt.

59. The proconstruct according to claim 58, wherein the distance between the last nucleotide of said insertion site and the first nucleotide of said terminator is less than about 5 nt.

60. The proconstruct according to claim 60, wherein the distance between the last nucleotide of said insertion site and the first nucleotide of said terminator ranges from about 0 to about 5 nt.

61. The proconstruct according to claim 55, wherein said insertion site is a multiple cloning site.

62. The proconstruct according to claim 55, wherein said insertion site comprises at least one sequence cleaved by a type IIS restriction endonuclease.

63. The proconstruct according to claim 55, wherein at least one of said two opposing promoters is an inducible promoter.

64. The proconstruct according to claim 55, wherein said proconstruct is a provector.

65. The proconstruct according to claim 64, wherein said provector is a plasmid provector.

66. The proconstruct according to claim 64, wherein said provector is a viral provector.

67. A kit for use in preparing an RNAi double-stranded molecule, said kit comprising:
(a) a proconstruct comprising said two opposing promoters flanking an insertion site, wherein each of said promoters comprises a transcription terminator, and
(b) instructions for inserting a siRNA coding domain into said insertion site.

68. The kit according to claim 67, wherein said transcription terminator is present in a non-transcribed region of each of said promoters.

69. The kit according to claim 68, wherein in each of said promoters, said transcription terminator is adjacent to said insertion site.

70. The kit according to claim 69, wherein the distance between the last nucleotide of said insertion site and the first nucleotide of said terminator is less than about 20 nt.

71. The kit according to claim 70, wherein the distance between the last nucleotide of said insertion site and the first nucleotide of said terminator is less than about 10 nt.

72. The kit according to claim 71, wherein the distance between the last nucleotide of said insertion stie and the first nucleotide of said terminator is less than about 5 nt.

73. The kit according to claim 72, wherein the distance between the last nucleotide of said insertion site and the first nucleotide of said terminator ranges from about 0 to about 5 nt.

74. The kit according to claim 67, wherein said kit further comprises a restriction endonuclease for said insertion site.

75. The kit according to claim 67, wherein said insertion site is a multiple cloning site.

76. The kit according to claim 67, wherein said insertion site comprises at least one sequence cleaved by a type IIS restriction endonuclease.

77. The kit according to claim 67, wherein at least one of said two opposing promoters is an inducible promoter.

78. A system for use in preparing an RNAi double-stranded molecule, said system comprising:
(a) a proconstruct comprising said two opposing promoters flanking an insertion site, wherein each of said promoters comprises a transcription terminator, and
(b) a restriction endonuclease for said cloning site.

79. The system according to claim 78, wherein said transcription terminator is present in a non-transcribed region of each of said promoters.

80. The system according to claim 79, wherein in each of said promoters, said transcription terminator is adjacent to said insertion site.

81. The system according to claim 80, wherein the distance between the last nucleotide of said insertion site and the first nucleotide of said terminator is less than about 20 nt.

82. The system according to claim 81, wherein the distance between the last nucleotide of said insertion site and the first nucleotide of said terminator is less than about 10 nt.

83. The system according to claim 82, wherein the distance between the last nucleotide of said insertion stie and the first nucleotide of said terminator is less than about 5 nt.

84. The system according to claim 83, wherein the distance between the last nucleotide of said insertion site and the first nucleotide of said terminator ranges from about 0 to about 5 nt.

85. The system according to claim 78, wherein said insertion site is a multiple cloning site.

86. The system according to claim 78, wherein said insertion site comprises at least one sequence cleaved by a type IIS restriction endonuclease.

87. The system according to claim 78, wherein at least one of said two opposing promoters is an inducible promoter.
